(19) United States
(12) Patent Application Publication
(10) Pub. No.: US 2024/0197963 A1
(43) Pub. Date: Jun. 20, 2024

(54) 3D-PRINTING OF STRONG LIVING SCAFFOLDS

(71) Applicant: Northwestern University, Evanston, IL (US)

(72) Inventors: Yonghui DING, Evanston, IL (US); Cheng SUN, Evanston, IL (US); Guillermo AMEER, Evanston, IL (US)

(21) Appl. No.: 18/556,311

(22) PCT Filed: Apr. 19, 2022
(86) PCT No.: PCT/US22/25359

§ 371 Date: Oct. 19, 2023

Related U.S. Application Data
(60) Provisional application No. 63/176,752, filed on Apr. 19, 2021.

(51) Int. Cl. A61L 27/52; A61L 27/26; A61L 27/38; A61L 27/54; A61N 5/06

(52) U.S. Cl. CPC A61L 27/52; A61L 27/26; A61L 27/3804; A61L 27/54; A61N 5/062; A61L 2300/414; A61L 2300/62; A61L 2300/802; A61N 2005/0661

(57) ABSTRACT

Provided herein are compositions and methods for 3D bioprinting of mechanically strong and biologically active scaffolds using emulsion bioink for the regeneration of a wide variety of tissues with biochemical functions (e.g., bone, tendon, ligament, cartilage, etc.).

### CROSS-REFERENCE TO RELATED APPLICATIONS

This application claims the benefit of U.S. Provisional Patent Application No. 63/176,752, filed on Apr. 19, 2022, which is incorporated by reference herein.

### FIELD

Provided herein are compositions and methods for 3D printing of mechanically strong and biologically active scaffolds using emulsion bioink for the regeneration of a wide variety of tissues with biochemical functions (e.g., bone, tendon, ligament, cartilage, etc.).

### BACKGROUND

The regeneration of damaged or diseased tissues that serve biomechanical functions, such as musculoskeletal tissues, has been a long-standing challenge in clinical practice and research. Regenerative engineering offers a promising alternative to auto- and allografts in tissue regeneration by combining biomaterial scaffolds, viable cells, and bioactive factors. Engineering scaffolds that provide both mechanical support and biological activities is critical for regenerating such tissues with biomechanical functions; however, it remains an enormous challenge. Currently existing scaffolds, falling into the categories of tough polymer scaffolds with limited bioactivities or hydrogels with poor mechanical properties (elastic modulus E<0.1 MPa), fall short of meeting both mechanical and biological needs. Existing methods to produce strong living scaffolds rely on the post-loading of cells into pre-fabricated strong scaffolds, which suffer from, for example, the non-uniform distribution of cells. Therefore, an effective strategy for fabricating strong living scaffolds is warranted.

### SUMMARY

Provided herein are compositions and methods for 3D printing of mechanically strong and biologically active scaffolds using emulsion bioink for the regeneration of a wide variety of tissues with biochemical functions (e.g., bone, tendon, ligament, cartilage, etc.).

In some embodiments, provided herein are bioink compositions comprising emulsifier-coated hydrogel microparticles dispersed within a polymer solution continuous phase comprising a photoinitiator. In some embodiments, compositions further comprise bioactive factors and/or cells encapsulated the hydrogel microparticles. In some embodiments, the hydrogel microparticles comprise gelatin methacrylate (GelMA), collagen methacrylate (ColMA), poly(ethylene glycol) diacrylate (PEDGA), cell-adhesive poly(ethylene glycol), MMP-sensitive poly(ethylene glycol), poly(ethylene glycol) dimethacrylate (PEGDMA), poly(ethylene glycol) diacrylamide (PEGDAAm), methacrylated hyaluronic acid (MeHA), PEGylated fibrinogen, and combinations thereof. In some embodiments, the hydrogel microparticles comprise methacrylated gelatin (GelMA). In some embodiments, the polymer solution continuous phase comprises epoxy vinyl ester prepolymers and polymers, diallyl phthalate (DAP), diallyl isophthalate (DAIP), triallyl isocyanurate, glycerol propoxylate triacrylatee (GPTA), trimethylolpropane triacrylatee (TMPTA), pentaerythritol diacrylatee mono stearate (PEAS), hexanediol diacrylatee (HDDA), 1,6-hexanediol ethoxylate diacrylate (HDEDA), hexanediol dimethacrylatee (HDDMA), hydrocortisone acrylate (HCNA), and combinations thereof. In some embodiments, the polymer solution continuous phase comprises 1,6-Hexanediol diacrylate (HDDA). In some embodiments, the emulsifier comprises sorbitan monooleate (SMO), sorbitan monolaurate (SML)), polyglycerol polyricinoleate (PGPR), polyglycerol polyricinoleate, a hydrophobic-hydrophilic block copolymer, Poloxamer 407, Triton X-405, Triton X-100, Triton X-705 Tween 20, polyglycerol polyricinoleate (PGPR), and any combination thereof. In some embodiments, the emulsifier comprises polyglycerol polyricinoleate (PGPR). In some embodiments, the photoinitiator comprises ethyl (2,4,5-trimethylbenzoyl) phenyl phosphinate (TPO-L), 2-hydroxy-2-methyl propiophenone, methylbenzoyl formate, isoamyl 4-(dimethylamino) benzoate, 2-ethyl hexyl-4-(dimethylamino) benzoate, or diphenyl(2,4,6-trimethylbenzoyl) phosphine oxide (TPO), phenylbis(2,4,6-trimethyl benzoyl)phosphine oxide (BAPO), and combinations thereof. In some embodiments, the photoinitiator is Phenylbis(2,4,6-trimethyl benzoyl)phosphine oxide (BAPO).

In some embodiments, the bioink comprises PGPR-coated GelMA microparticles dispersed within a HDDA continuous phase. In some embodiments, the bioink further comprises a BAPO photoinitiator. In some embodiments, the bioink further comprises bioactive factors and/or cells encapsulated within the GelMA microparticles. In some embodiments, the bioink further comprises cells selected from fibroblasts, macrophages, mast cells, osteoblasts, osteocytes, osteoclasts and/or bone lining cells. In some embodiments, the bioactive factors comprise growth factors

In some embodiments, provided herein are scaffolds (e.g., 2D or 3D cellular scaffolds) produced by exposing a bioink composition herein to initiator conditions (e.g., ultraviolet light). In some embodiments, methods further comprise depositing the bioink into a 2D or 3D orientation by microscale continuous liquid interface production (CLIP).

In some embodiments, provided herein are methods of tissue repair, growth, or regeneration comprising implanting a scaffold described herein into a subject (e.g., at the site of a tissue injury). In some embodiments, provided herein are methods of tissue repair, growth, or regeneration comprising placing a bioink described herein into a subject (e.g., at the site of a tissue injury), exposing the bioink to conditions that allow for formation of a solid scaffold. In some embodiments, conditions that allow for formation of a solid scaffold comprise UV light.

### BRIEF DESCRIPTION OF THE FIGURES

FIG. 1. Schematic of emulsion bioink and mechanism by which living cells are protected.

FIG. 2A-B. Preparation and cytocompatibility of exemplary emulsion bioink. (A) Schematic of preparation method, photograph, and optical image of GelMA/HDDA emulsion bioink. (B) Quantification of cell viability and representative confocal image of fibroblasts in emulsion bioink after incubation for 2 hours.

FIG. 3. An exemplary prepared emulsion bioink is 3D-printable. SEM images of lyophilized polymer scaffold with smooth surface (left) and emulsion scaffold containing microgel particles on polymer matrix (middle). Confocal images (right) of hydrated emulsion scaffold reveal the formation of GelMA microgel particles by labelling the GelMA with a fluorescent dye.

FIG. 4A-B. 3D-printed emulsion scaffolds are mechanically strong and biologically active. (A) Emulsion scaffolds are 333-2667 times stronger than hydrogel (E=15 KPa) and their compressive moduli (5-40 MPa) are tunable by varying the loading concentrations of HDDA polymer in emulsion. (B) Fibroblast cells that are trapped in emulsion scaffolds maintain >90% viability for at least 7 days.

### DETAILED DESCRIPTION

Provided herein are compositions and methods for 3D printing of mechanically strong and biologically active scaffolds using emulsion bioink for the regeneration of a wide variety of tissues with biochemical functions (e.g., bone, tendon, ligament, cartilage, etc.). In particular embodiments provided herein are water-in-oil emulsion bioinks, and the preparation thereof by dispersing hydrogel microparticles (microgel, dispersed phase) with entrapped bioactive factors and/or cells within a tough polymer solution (continuous phase). In some embodiments, provided herein are living scaffolds and methods of preparation thereof by 3D printing of the emulsion bioinks herein.

Other existing scaffolds, either hydrogels or tough polymers, fall short of meeting both mechanical and biological needs for tissue regeneration. Existing methods to produce living scaffolds rely on the post-loading of cells into pre-fabricated scaffolds, which suffer from, for example, the non-uniform distribution of cells. The compositions and methods herein overcome these deficiencies. Embodiments herein enable the direct encapsulation and effective delivery of living cells and/or bioactive factors within tough polymer scaffolds. The strong living scaffolds have a broad and significant impact on the repair or regeneration of broad-spectrum tissues with biomechanical functions, such as bone, tendon, ligament, and cartilage.

In some embodiments, provided herein are bioink compositions comprising emulsifier-coated hydrogel microparticles dispersed within a tough polymer solution as a continuous phase comprising a photoinitiator. In some embodiments, bioactive factors and/or cells are encapsulated within the hydrogel microparticles.

In some embodiments, the compositions herein comprise a hydrogel (e.g., hydrogel microparticles). The dispersed phase hydrogel microparticles provide biocompatible environments to encapsulated bioactive factors and/or cells. In some embodiments, the hydrogel microparticles comprise gelatin methacrylate (GelMA), collagen methacrylate (ColMA), poly(ethylene glycol) diacrylate (PEDGA), cell-adhesive poly(ethylene glycol), MMP-sensitive poly(ethylene glycol), poly(ethylene glycol) dimethacrylate (PEGDMA), poly(ethylene glycol) diacrylamide (PEGDAAm), methacrylated hyaluronic acid (MeHA), PEGylated fibrinogen, and combinations thereof. In particular embodiments, the hydrogel microparticles comprise methacrylated gelatin (GelMA). In some embodiments, the hydrogel comprises a methacrylated or acrylated polymer.

In some embodiments, the compositions herein comprise a continuous phase tough polymer (or monomers or pre-polymer thereof). The continuous phase polymer encapsulates the hydrogel microparticles and forms a external phase contributing to the mechanical properties of a resulting scaffold. Suitable continuous phase polymers (or monomers or pre-polymers thereof) are selected from epoxy vinyl ester prepolymers and polymers, diallyl phthalate (DAP), diallyl isophthalate (DAIP), triallyl isocyanurate, glycerol propoxylate triacrylatee (GPTA), trimethylolpropane triacrylatee (TMPTA), pentaerythritol diacrylatee mono stearate (PEAS), hexanediol diacrylatee (HDDA), 1,6-hexanediol ethoxylate diacrylate (HDEDA), hexanediol dimethacrylatee (HDDMA), hydrocortisone acrylate (HCNA), and combinations thereof. In some embodiments, the continuous phase polymer (or monomers or pre-polymer thereof) is photosensitive. In some embodiments, polymerization is initiated by exposure to free radicals. In particular embodiments, the polymer solution continuous phase comprises 1,6-Hexanediol diacrylate (HDDA).

In some embodiments, the compositions herein comprise an emulsifier and/or surfactant. An emulsifier and/or surfactant functions as a protective shield at the interface to protect encapsulated bioactive factors and/or cells by limiting the chemical diffusion from polymeric continuous phase into hydrogel microparticles. In addition, an emulsifier and/or surfactant stabilizes the emulation of the hydrogel microparticles with the polymeric continuous phase. Suitable emulsifiers/surfactants may be selected from the Span family of surfactants (such as sorbitan monooleate (SMO), sorbitan monolaurate (SML)), polyglycerol polyricinoleate (PGPR), and the Hypermer family of surfactants. In some embodiments, an emulsifier/surfactant is selected from the group consisting of sorbitan monooleate, polyglycerol polyricinoleate, a hydrophobic-hydrophilic block copolymer, and any combination thereof. In some embodiments, the emulsifier/surfactant is Poloxamer 407, Triton X-405, Triton X-100, Triton X-705 and Tween 20. In particular embodiments, the emulsifier comprises polyglycerol polyricinoleate (PGPR).

In some embodiments, the compositions herein comprise a free-radical initiator. A function of the free radical initiator is to generate free radicals that initiate the formation of the polymeric network within the continuous phase polymer. Non-limiting examples of free radical initiators includes ammonium persulfate (APS), ribofalvin-5′-phosphate, ribofalvin-5′-phosphate sodium, peroixdes such as dialkyl peroxides, hydroperoxides, diacyl periods, or azo-compounds (i.e., --N.dbd.N-- moieties). In some embodiments, the free radical initiator is a photoinitiator. Non-limiting examples of photoinitiators include ethyl (2,4,5-trimethylbenzoyl) phenyl phosphinate (TPO-L), bis-acylphosphine oxide (BAPO), 2-hydroxy-2-methyl propiophenone, methylbenzoyl formate, isoamyl 4-(dimethylamino) benzoate, 2-ethyl hexyl-4-(dimethylamino) benzoate, or diphenyl(2,4,6-trimethylbenzoyl) phosphine oxide (TPO). Additional, non-limiting examples of suitable photo-initiators include 1-hydroxycyclohexyl phenyl ketone (Irgacure 184), 2,2-dimethoxy-2-phenylacetophenone (Irgacure 651), and 2-methyl-1-[4-(methylthio) phenyl]-2-(4-morpholinyl)-1-propanone (Irgacure 907), hydroxyacetophenone, phosphineoxide, benzophenone, and lithium phenyl-2,4,6-trimethylbenzoylphosphinate (LAP). In some embodiments, the free-radical initiator is a photoinitiator and comprises phenylbis(2,4,6-trimethyl benzoyl)phosphine oxide (BAPO). In some embodiments, the free-radical initiator (e.g., photoinitiator) is present in a composition herein at 0.01 wt %, 0.02 wt %, 0.05 wt %, 0.1 wt %, 0.2 wt %, 0.5 wt %, 1 wt %, or ranges therebetween.

In some embodiments, provided herein are exemplary bioink compositions comprising PGPR-coated GelMA microparticles dispersed within a HDDA continuous phase comprising a BAPO photoinitiator.

In some embodiments, the bioink compositions herein comprise bioactive factors and/or cells encapsulated within the GelMA microparticles.

In some embodiments, the compositions and scaffolds herein comprise one or more bioactive factors or agents contained therein (e.g., encapsulated within the hydrogel microparticles). Suitable bioactive factors and/or agents include therapeutic molecules (e.g., drugs), biological macromolecules (e.g., peptides, nucleic acids, proteins, lipids, etc.), cofactors, cytokines, growth factors, etc.

In some embodiments, the compositions and scaffolds herein comprise one or more bioactive factors or agents selected from therapeutic compounds, such as an antibacterial, antiviral, antifungal or antiparasitic compound, cytotoxic or anti-cancer compound; an immune stimulatory or inhibitor agent; a pro-angiogenic factor; an anti-inflammatory agent; an anti-helminth; an antihistamine; an anticoagulant; a beta-adrenergic receptor inhibitor; a calcium channel blocker; an ace inhibitor; etc.

In some embodiments, the compositions and scaffolds herein comprise (e.g., encapsulated within the hydrogel microparticles) one or more bioactive factors or agents selected from protein/polypeptide agents, such as an enzyme, a receptor, a channel protein, a hormone, a cytokine, a growth factor, and antibody drug. In some embodiments, the materials described herein encapsulate and/or find use in the delivery of growth factors for the repair of tissue/bone defects and/or generation/regeneration of tissue/bone. Suitable bioactive factors include bone morphogenic proteins (e.g., BMP-1, BMP-2, BMP-4, BMP-6, and BMP-7); members of the transforming growth factor beta (TGF-β) superfamily including, but not limited to, TGF-β1, TGF-β2, and TGF-β3; epidermal growth factor (EGF), transforming growth factor-alpha (TGF-α), growth differentiation factors (GDF1, GDF2, GDF3, GDF5, GDF6, GDF7, myostatin/GDF8, GDF9, GDF10, GDF11, and GDF15); human endothelial cell growth factor (ECGF); granulocyte macrophage colony stimulating factor (GM-CSF); nerve growth factor (NGF); vascular endothelial growth factor (VEGF); fibroblast growth factor (FGF); insulin-like growth factor (IGF); cartilage derived morphogenetic protein (CDMP); platelet rich plasma (PRP); platelet derived growth factor (PDGF); cartilage-derived morphogenetic protein (CDMP); insulin growth factor one (IGF-I); platelet-derived growth factor (PDGF); or any combinations thereof.

In some embodiments, the compositions and scaffolds herein comprise (e.g., encapsulated within the hydrogel microparticles) one or more bioactive factors or agents selected from natural or non-natural insulins, amylases, proteases, lipases, kinases, phosphatases, glycosyl transferases, trypsinogen, chymotrypsinogen, carboxypeptidases, hormones, ribonucleases, deoxyribonucleases, triacylglycerol lipase, phospholipase A2, elastases, amylases, blood clotting factors, UDP glucuronyl transferases, ornithine transcarbamoylases, cytochrome p450 enzymes, adenosine deaminases, serum thymic factors, thymic humoral factors, thymopoietins, growth hormones, somatomedins, costimulatory factors, antibodies, colony stimulating factors, erythropoietin, epidermal growth factors, hepatic erythropoietic factors (hepatopoietin), liver-cell growth factors, interleukins, interferons, negative growth factors, fibroblast growth factors, transforming growth factors of the .alpha. family, transforming growth factors of the .beta. family, gastrins, secretins, cholecystokinins, somatostatins, serotonins, substance P, transcription factors or combinations thereof.

In some embodiments, the compositions and scaffolds herein comprise (e.g., encapsulated within the hydrogel microparticles) one or more bioactive factors or agents selected from nucleic acids, such as DNA or vectors encoding a gene or an inhibitory RNA (e.g., siRNA, shRNA, antisense RNA, CRISPR RNA, etc.).

In some embodiments, the compositions and scaffolds herein comprise (e.g., encapsulated within the hydrogel microparticles) cells. In some embodiments, cells are provided within the materials herein for delivery and/or use in tissue repair, regeneration, generation, etc. applications. In some embodiments, cells encapsulated within the bioink compositions herein are selected from fibroblasts, chondrocytes, macrophages, mast cells, osteoblasts, osteocytes, osteoclasts, bone lining cells, and/or other bone cartilage, ligament, tendon, etc. tissues.

In some embodiments, the compositions and scaffolds herein find use in the delivery of cells and/or bioactive agents for tissue repair, growth, and/or regeneration applications. In some embodiments, the compositions herein (e.g., made from the bioactive inks herein) find use as scaffolds for cell and/or tissue growth. In some embodiments, materials herein find use in growth and/or repair of tissues, such as bone, ligaments, cartilage, tendon, etc. In some embodiments, compositions and scaffolds herein find use growth or repair of hyaline cartilage (e.g., costal cartilages, the cartilages of the nose, trachea, and bronchi, and the articular cartilages of joints), elastic cartilage (e.g., external ear, external auditory meatus, part of the Eustachian tube, epiglottis, and in some of the laryngeal cartilages) and/or fibrocartilage (e.g. meniscus (e.g., wrist triangular fibrocartilage complex, knee meniscus), intervertebral discs, temporomandibular joint disc, the pubic symphysis, etc.).

In some embodiments, methods are provided for preparation of a bioink material comprising a hydrogel, surfactant and/or emulsifier, and a polymer continuous phase. In some embodiments, an aqueous phase comprising the hydrogel is added to the polymer continuous phase (or vice versa) in the present of the emulsifier. In some embodiments, stirring of the three components results in emulsification and encapsulation of the hydrogel within the surfactant and/or emulsifier in the polymer continuous phase. In some embodiments, the aqueous phase further comprises one or more types of cells and/or bioactive agents/factors. In some embodiments, the aqueous phase and/or the polymer continuous phase further comprises a free radical initiator (e.g., photoinitiator).

In some embodiments, methods are provided for the formation (e.g., printing) of 2D or 3D scaffolds from the bioinks described herein. In some embodiments, a bioink described herein is formed into a desired geometry (2D or 3D) and then exposed to a condition (e.g., UV light) to cause a free radical initiator (e.g., photoinitiator) to induce polymerization of the bioink into a solid scaffold. In some embodiments, the bioink is used in a 3D printing process. 3D printing, also known as additive manufacturing (AM), is a term used to describe several different processes that builds a user-designed CAD part layer-by-layer until completion (Giannatsis, J. and V. Dedoussis, The International Journal of Advanced Manufacturing Technology, 40(1-2): 116-127 (2009)). These processes include photopolymerization methods, extrusion-based methods, laser-induced melting/sintering, etc. 3D printing techniques give the designer geometric flexibility that is troublesome for standard subtractive manufacturing processes (Giannatsis, J. and V. Dedoussis, The International Journal of Advanced Manufacturing Technology, 40(1-2): 116-127 (2009); Melchels et al., Biomaterials, 31(24): 6121-6130 (2010)). 3D printing has typically been used for small batch manufacturing, such as prototype manufacturing and biomedicine for patient specific needs.

Continuous liquid interface processing (CLIP) is an additive manufacturing process that utilizes photopolymerization to create 3D geometric parts. CLIP could be considered a 3rd generation of stereolithography AM process. Projection stereolithography (PSL; stereolithography 2nd generation) utilizes patterning the UV light via a dynamic mask generator to allow fabrication of each cross-sectional layer in a single exposure (Sun et al., Sensors and Actuators A: Physical, 121(1): 113-120 (2005)). In-plane resolution of PSL is dependent on the pixel size of the dynamic mask generator. In the case of projection microstereolithography (PuSL), in-plane resolution can be sub-20 μm. With an emphasis on high precision and surface finish, a high resolution microCLIP process has recently been developed (van Lith, et al. 3D-Printing Strong High Resolution Antioxidant Bioresorbable Vascular Stents). Advanced Materials Technologies, 2016). CLIP/microCLIP works in a similar manner to PSL with the addition of an air/oxygen permeable window placed between the UV light optics and the photopolymer resin. Oxygen is a natural inhibitor of the photopolymerization reaction. With introduction of oxygen permeable window into the UV light pathway allows a small region where no polymerization occurs (Tumbleston et al., Science, 347(6228): 1349-1352 (2015)). This allows removal of a “delamination” step during fabrication (removing polymerized layer from optical window). In addition, this small polymerization-free region allows new photoresin to flow in and replace polymerized material, allowing essentially continuous fabrication of each layer onto the previous layer until the part is complete. CLIP/microCLIP has caused a massive reduction in fabrication time within the stereolithography processes, which typically range from several hours to now several minutes (van Lith et al., supra, and Tumbleston et al., supra). This process has opened the door to newer materials that may have been troublesome to utilize in previous stereolithography techniques, such as very viscous materials or materials that need quickly evaporating solvents.

In some embodiments, scaffolds produced by the methods herein are implanted into a subject (e.g., at a location in need of tissue or bone repair, at the site of tissue or bone injury). In some embodiments, scaffolds produced by the methods herein are used to deliver cells and/or bioactive agents to a subject (e.g., to tissue and/or bone of a subject). In some embodiments, scaffolds produced by the methods herein are used for the growth of cells.

### EXPERIMENTAL

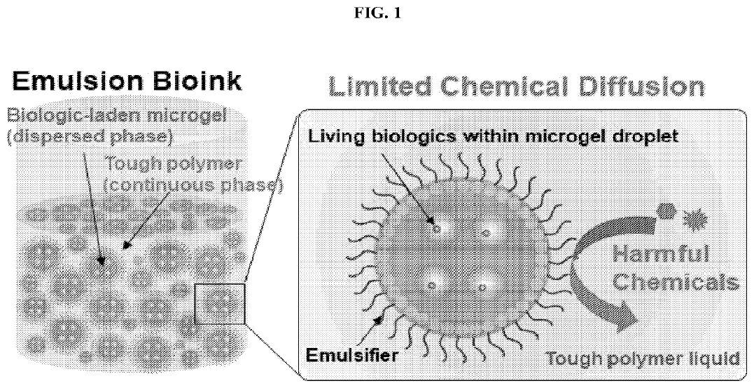

Experiments were conducted during development of embodiments herein to develop a water-in-oil emulsion bioink (FIG. 1) in which an internal phase of hydrogel droplets (microgels) comprising encapsulated living cells and/or biological factors are dispersed in an external phase of tough polymer to enable the production of strong living scaffolds. The emulsion protects living cells and biological factors within microgels from harmful chemicals in tough polymer liquid by limiting chemical diffusion. The photo-polymerization of the external tough polymer around each internal microgel during 3D-printing contributes to the mechanical robustness of the final scaffold.

### Synthesis of Methacrylated Gelatin (GelMA)

GelMA was synthesized as described previously (Nichol et al. Biomaterials. 2010, 31: 5536-5544; incorporated by reference in its entirety). 5 g type A porcine skin gelatin (MilliporeSigma, Billerica, MA) was mixed at 10% (w/v) into Dulbecco's phosphate buffered saline (DPBS) (GIBCO) at 60° C. and stirred until fully dissolved. 4 mL Methacrylic anhydride (MA, MilliporeSigma) was added at a rate of 0.5 mL/min to the gelatin solution under stirred conditions at 50° C. and allowed to react for 3 hours. The fraction of lysine groups reacted was modified by varying the amount of MA present in the initial reaction mixture. Following a 5× dilution with additional warm (40° C.) DPBS to stop the reaction, the mixture was dialyzed against distilled water using 12-14 kDa cut-off dialysis tubing for 1 week at 40° C. to remove salts and methacrylic acid. The solution was lyophilized for 1 week to generate a white porous foam and stored at −20° C. until further use.

### Preparation and Cytocompatibility of Exemplary Emulsion Bioink

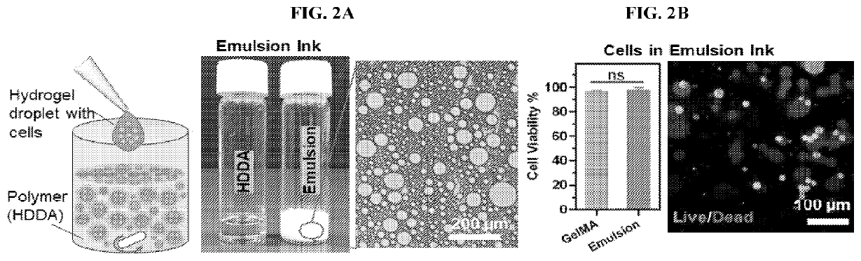

The 0.2 mL 1,6-Hexanediol diacrylate (HDDA) (MilliporeSigma) pre-polymer solution with 0.1 wt % photoinitiator Phenylbis(2,4,6-trimethyl benzoyl)phosphine oxide (BAPO or 1819 from MilliporeSigma) and 10 wt % emulsifier polyglycerol polyricinoleate (PGPR 4150) (Palsgaard, Denmark) was added to a 5 mL glass bottle. The pre-polymer solution was stirred continually at 400 rpm using a magnetic stirrer at room temperature. An aqueous phase consisting of 1.5×10⁶ cells (L929 mouse fibroblasts) (ATCC, USA) in 0.3 mL either DPBS or 6 wt % GelMA with 5 mM photoinitiator lithium phenyl-2,4,6-trimethyl-benzoyl phosphinate (LAP) (TCI America, Portland, OR) in DPBS was added dropwise (˜15 μL per drop) over a period of two minutes into the HDDA pre-polymer solution. After addition of the aqueous phase was complete, the emulsion bioink was stirred for another 10 minutes. The successful formation of emulsion was indicated by the color change of the solution from clear to opaque as well as the observation of dispersed hydrogel droplets under an optical microscope (FIG. 2A).

To evaluate the cytocompatibility of the emulsion bioink, mouse fibroblast cells were suspended in GelMA solution containing LIVE/DEAD Viability/Cytotoxicity Kit (ThermoFisher, USA) following the manufacturer's instruction and incubated for 15 min prior to being added to HDDA pre-polymer solution to make the emulsion bioink. The prepared emulsion bioink was incubated at 37° C. in an incubator for 2 hours and fluorescent images were acquired under a spinning-disk confocal microscope (Leica). The results showed that >95% cell viability was preserved in emulsion bioink, which was similar to pure GelMA, suggesting the good cytocompatibility of the emulsion bioink (FIG. 2B).

### The Prepared Emulsion Bioink is 3D-Printable.

The prepared emulsion bioink was utilized to fabricate the emulsion scaffolds using a custom-made microscale continuous liquid interface production (CLIP) 3D printing system, which utilized ultraviolet (UV) light (λ=365 nm) for photopolymerization and ambient air as the oxygen source for establishing the “dead-zone”. The CAD design of scaffolds was sliced with a layer slice thickness between 5 and 15 μm. The UV power density of 5.8 mW/cm² and exposure time of 0.2 s per layer were used to produce scaffolds. The 3D-printed emulsion scaffolds were rinsed in a large amount of DPBS solution (˜6-7 mL) twice and then transferred to 6 mL growth media consisting of Eagle's Minimum Essential Medium (EMEM) (Lonza, Rockland, ME) with 10% fetal bovine serum (FBS). The growth media was refreshed in 1 hour and 3 hours, respectively.

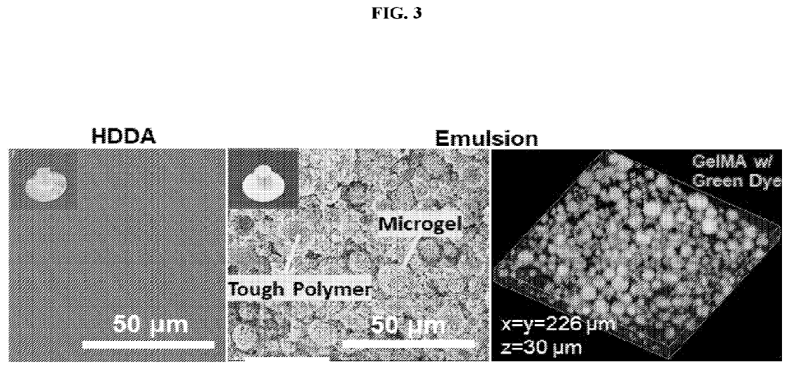

The acellular emulsion scaffolds were lyophilized overnight, coated with a 10 nm layer of Au/Pt, and observed using a scanning electron microscope (SEM). It was observed that porous GelMA microgel particles were embedded on HDDA polymer matrix throughout the scaffolds (FIG. 3, middle). In addition, the hydrated emulsion scaffolds were observed under a confocal microscope. The confocal images showed the successful formation of GelMA microgels, which was labelled by a green-colored dye, within emulsion scaffolds (FIG. 3, right).

### The 3D-Printed Emulsion Scaffolds are Mechanically Strong and Biologically Active.

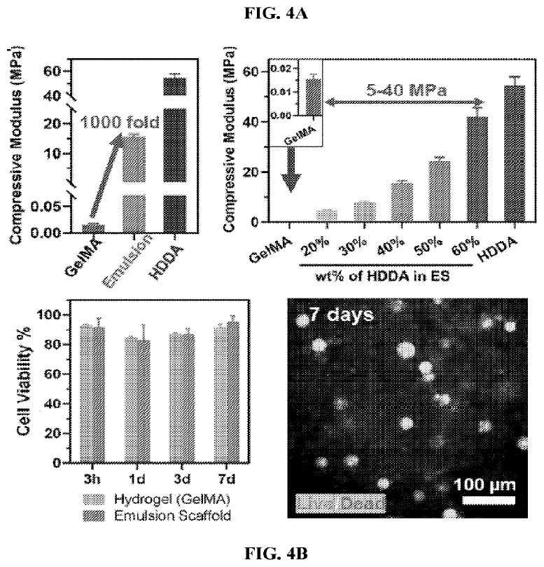

The mechanical property of emulsion scaffolds was evaluated by compression test using Instron universal tester (Model 5940, Instron, High Wycombe, UK) equipped with a 2 kN load cell. The scaffolds were placed between two parallel plates and compressed at a crosshead displacement speed of 1 mm/min until failure, which conformed to ASTM Standard D3410. The results showed that the compression moduli of fully hydrated emulsion scaffolds can be tuned over an order of magnitude (5-40 MPa), which are 333-2667 times stronger than GelMA hydrogels (E=15 KPa), by varying the loading concentrations of HDDA polymer in emulsion (FIG. 4A). They recapitulate moduli of many tissues, like meniscus, articular cartilage, trachea, temporomandibular disc etc. Moreover, the cell-laden emulsion scaffolds were incubated with LIVE/DEAD Viability/Cytotoxicity Kit for 30 min and imaged under confocal microscope. The results showed that the encapsulated fibroblast cells were maintained >85-90% viability for at least 7 days (FIG. 4B).

1. A bioink composition comprising emulsifier-coated hydrogel microparticles dispersed within a polymer solution continuous phase comprising a photoinitiator.

2. The bioink composition of claim 1, further comprising bioactive factors and/or cells encapsulated within the hydrogel microparticles.

3. The bioink composition of claim 1, wherein the hydrogel microparticles comprise gelatin methacrylate (GelMA), collagen methacrylate (ColMA), poly(ethylene glycol) diacrylate (PEDGA), cell-adhesive poly(ethylene glycol), MMP-sensitive poly(ethylene glycol), poly(ethylene glycol) dimethacrylate (PEGDMA), poly(ethylene glycol) diacrylamide (PEGDAAm), methacrylated hyaluronic acid (MeHA), PEGylated fibrinogen, and combinations thereof.

4. The bioink composition of claim 3, wherein the hydrogel microparticles comprise methacrylated gelatin (GelMA).

5. The bioink composition of claim 1, wherein the polymer solution continuous phase comprises epoxy vinyl ester prepolymers and polymers, diallyl phthalate (DAP), diallyl isophthalate (DAIP), triallyl isocyanurate, glycerol propoxylate triacrylatee (GPTA), trimethylolpropane triacrylatee (TMPTA), pentaerythritol diacrylatee mono stearate (PEAS), hexanediol diacrylatee (HDDA), 1,6-hexanediol ethoxylate diacrylate (HDEDA), hexanediol dimethacrylatee (HDDMA), hydrocortisone acrylate (HCNA), and combinations thereof.

6. The bioink composition of claim 5, wherein the polymer solution continuous phase comprises 1,6-Hexanediol diacrylate (HDDA).

7. The bioink composition of claim 1, wherein the emulsifier comprises sorbitan monooleate (SMO), sorbitan monolaurate (SML)), polyglycerol polyricinoleate (PGPR), polyglycerol polyricinoleate, a hydrophobic-hydrophilic block copolymer, Poloxamer 407, Triton X-405, Triton X-100, Triton X-705 Tween 20, polyglycerol polyricinoleate (PGPR), and any combination thereof.

8. The bioink composition of claim 7, wherein the emulsifier comprises polyglycerol polyricinoleate (PGPR).

9. The bioink composition of claim 1, wherein the photoinitiator comprises ethyl (2,4,5-trimethylbenzoyl) phenyl phosphinate (TPO-L), 2-hydroxy-2-methyl propiophenone, methylbenzoyl formate, isoamyl 4-(dimethylamino) benzoate, 2-ethyl hexyl-4-(dimethylamino) benzoate, or diphenyl(2,4,6-trimethylbenzoyl) phosphine oxide (TPO), phenylbis(2,4,6-trimethyl benzoyl)phosphine oxide (BAPO), and combinations thereof.

10. The bioink composition of claim 9, wherein the photoinitiator is Phenylbis(2,4,6-trimethyl benzoyl)phosphine oxide (BAPO).

11. The bioink composition of claim 1, comprising PGPR-coated GelMA microparticles dispersed within a HDDA continuous phase.

12. The bioink composition of claim 11, further comprising BAPO photoinitiator.

13. The bioink composition of claim 11, further comprising bioactive factors and/or cells encapsulated within the GelMA microparticles.

14. The bioink composition of claim 13, comprising cells selected from fibroblasts, macrophages, mast cells, osteoblasts, osteocytes, osteoclasts and/or bone lining cells.

15. The bioink composition of claim 13, comprising the bioactive factors comprise growth factors

16. A scaffold produced by exposing a bioink composition of one of claims 1-15 to ultraviolet light.

17. The scaffold of claim 16, further comprising depositing the bioink into a 2D or 3D orientation by microscale continuous liquid interface production (CLIP).

18. A method of tissue regeneration comprising implanting a scaffold of claim 16 or 17 into a subject.

19. A method of tissue growth or repair comprising placing a bioink of one of claims 1-15 into a subject, exposing the bioink to conditions that allow for formation of a solid scaffold.

20. The method of tissue growth or repair, wherein conditions that allow for formation of a solid scaffold comprise UV light.
